(19) United States
(12) Patent Application Publication
(10) Pub. No.: US 2017/0088587 A1
(43) Pub. Date: Mar. 30, 2017

(54) ANTIVIRAL FUSION PROTEINS AND GENES

(71) Applicant: Agenovir Corporation, South San Francisco, CA (US)

(72) Inventor: Stephen R. Quake, Stanford, CA (US)

(21) Appl. No.: 15/277,688

(22) Filed: Sep. 27, 2016

Related U.S. Application Data
(60) Provisional application No. 62/234,365, filed on Sep. 29, 2015.

(51) Int. Cl. C07K 14/005; C07K 14/195; C12N 9/22

(52) U.S. Cl. CPC C07K 14/005; C12N 9/22; C12Y 301/21004; C07K 14/195; C07K 2319/80; A61K 38/00

(57) ABSTRACT

Viral infection is a persistent cause of human disease. Fusion polypeptide systems target the genomes of viral infections, rendering the viruses incapacitated.

### RELATED APPLICATION

This application claims the benefit of U.S. Provisional Patent Application Ser. No. 62/234,365, filed Sep. 29, 2015, incorporated by reference.

### TECHNICAL FIELD

The invention generally relates to fusion polypeptides and their role in treating or eliminating latent viral infection.

### BACKGROUND

Viral infections are a significant medical problem. Various antiviral treatments are available but they generally are directed to interrupting the replicating cycle of the virus. Thus, a particularly difficult problem is latent viral infection, as there is no effective treatment to eradicate the virus from host cells. Since latent infection can evade immune surveillance and reactivate the lytic cycle at any time, there is a persistent risk throughout the life.

One example of a latent viral infection that is a particular problem is the herpesviridae virus family. Herpes viruses are some of the most widespread human pathogens, with more than 90% of adults having been infected with at least one of the eight subtypes of herpes virus. Latent infection persists in most people; and about 16% of Americans between the ages of 14 and 49 are infected with genital herpes, making it one of the most common sexually transmitted diseases. Due to latency, there is no cure for genital herpes or for herpes simplex virus type 2 (HSV-2). Once infected, a host carries the herpes virus indefinitely, even when not expressing symptoms. The Epstein-Barr virus (EBV), also called human herpesvirus 4 (HHV-4) is another member of the herpesviridae family and a common latent virus in humans. Epstein-Barr is known as the cause of infectious mononucleosis (glandular fever), and is also associated with particular forms of cancer, such as Hodgkin's lymphoma, Burkitt's lymphoma, nasopharyngeal carcinoma, and conditions associated with human immunodeficiency virus (HIV) such as hairy leukoplakia and central nervous system lymphomas. During latency, the EBV genome circularizes and resides in the cell nucleus as episomes. To date, however, no EBV vaccine or treatment exists. Similarly, human papillomavirus, or HPV, is a common virus in the human population, where more than 75% of women and men will have this type of infection at one point in their life. High-risk oncogenic HPV types are able to integrate into the DNA of the cell that can result in cancer, specifically cervical cancer. As with the herpesviridae family, due to the latent nature of HPV, no cure has been found.

Efforts have been made to develop drugs that target viral proteins but those efforts have not been wholly successful due to the latent nature of the viruses. For example, when a virus is in its latent state, it is not actively expressing its proteins, and thus there is nothing to target. Additionally, any effort to eradicate a viral infection is not useful if it interferes with host cellular function. For example, an enzyme that prevents viral replication is not helpful if it also interferes with replication in cells throughout the host. Accordingly, there exists a need to develop an effective means for treating these latent viruses.

### SUMMARY

The invention provides compositions and methods for selectively treating viral infections, including latent viral infections, using compositions that comprise a specific viral binding moiety linked to a polypeptide that cuts nucleic acid. Compositions and methods of the invention are useful to remove viral genetic material from a host organism, without interfering with the integrity of the host's genetic material. Compositions may be specifically targeted to remove only the viral nucleic acid without acting on host material either when the viral nucleic acid exists as a particle within the cell or when it is integrated into the host genome. Targeting the viral nucleic acid is preferably accomplished using a sequence-specific targeting polypeptide that targets viral genomic material for destruction by a cleaving polypeptide but does not target the host cell genome.

In a preferred embodiment, the cleaving polypeptide is the cleavage domain of a nuclease and the sequence-specific targeting polypeptide is a viral protein. In a further embodiment, the cleaving polypeptide is the cleavage domain of FokI and the target polypeptide is EBNA1. EBNA1 is an EBV viral protein that serves to localize the cleavage domain of FokI to a viral target sequence, wherein the cleavage domain of FokI cleaves DNA in a non-specific manner near the targeted sequence, causing breaks in the viral genome. Other targeting polypeptides can be used including, for example, the binding domains of deactivated clustered regularly interspaced short palindromic repeat (CRISPR)-associated nuclease (Cas9) or homologs thereof, hi-fi Cas9, Cpf1, argounate, PfAgo, NgAgo, zinc finger nucleases, transcription activator-like effector nucleases (TALENs), meganucleases, or any other system that can be used to target viral nucleic acid for cleavage by the cleaving polypeptide, such that the viral nucleic acid is degraded without interfering with the regular function of the host's genetic material. The cleaving polypeptide can make one or more single or double stranded breaks in the viral nucleic acid.

Compositions of the invention may be used to target viral nucleic acid in any form or at any stage in the viral life cycle. For example, the composition can digest viral RNA or DNA. In one embodiment, the viral infection is latent and the viral nucleic acid is integrated into the host genome. The host may be a living subject such as a human patient and the steps may be performed in vivo. Any suitable viral nucleic acid may be targeted for cleavage and digestion. In certain embodiments, the targeted viral nucleic acid can include, but is not limited to, nucleic acid from one or more viruses of the herpesviridae family, such as herpes simplex virus (HSV)-1, HSV-2, varicella zoster virus (VZV), Epstein-Barr virus (EBV), cytomegalovirus (CMV), human herpesvirus (HHV)-6A and -6B, HHV-7, and Kaposi's sarcoma-associated herpesvirus (KSHV), as well as nucleic acid from other viruses such as the human papillomavirus (HPV).

The cleaving polypeptide and the sequence-specific targeting polypeptide may be introduced into the cell using a vector. Vectors are typically categorized as viral or non-viral (e.g. plasmids). Suitable viral vectors may be, but are not limited to, for example, retrovirus, lentivirus, adenovirus, herpesvirus, poxvirus, alphavirus, vaccinia virus or adeno-associated viruses. Suitable non-viral vectors may include, but are not limited to, for example, a nanoparticle, a cationic lipid, a cationic polymer, metallic nanoparticle, a nanorod, a liposome, microbubbles, a cell-penetrating peptide, a liposphere and polyethyleneglycol (PEG). The cell may be prompted to take up the vector by, e.g., ultrasound or electroporation.

Aspects of the invention provide a composition for treatment of a viral infection. The composition includes a cleaving polypeptide and a sequence-specific targeting polypeptide that targets the composition to viral nucleic acid in vivo within a host cell thereby causing the cleaving polypeptide to cleave the viral nucleic acid. In certain embodiments, the cleaving polypeptide can be the cleavage domain of a nuclease and the sequence-specific binding can be a viral protein that specifically targets a portion of a viral genome. In one embodiment, the cleaving polypeptide is the cleavage domain of FokI and the targeting polypeptide is EBNA1.

In some aspects, the invention provides a composition for treatment of a viral infection including nucleic acid that encodes a cleaving polypeptide and a sequence-specific targeting polypeptide that targets the cleaving polypeptide to viral nucleic acid thereby causing the cleaving polypeptide to cleave the viral nucleic acid. The nucleic acid may comprise mRNA. In one embodiment, the cleaving polypeptide is the cleavage domain of FokI and the targeting polypeptide is EBNA1. In one aspect, the nucleic acid is provided within a delivery vector which may be a viral vector such as an adeno-associated virus. The vector can also include any of retrovirus, lentivirus, adenovirus, herpesvirus, poxvirus, alphavirus, vaccinia virus, a nanoparticle, a cationic lipid, a cationic polymer, a metallic nanoparticle, a nanorod, a liposome, microbubbles, cell-penetrating peptide, a liposphere, or polyethyleneglycol (PEG).

Compositions of the invention may be used to deliver a fusion polypeptide to a cell (including entire tissues) that is infected by a virus. It is to be understood that the term fusion polypeptide includes any composition that links a cleaving polypeptide to a targeting polypeptide in any manner. The fusion polypeptides are preferably designed to target viral nucleic acid. In some embodiments, the targeted viral nucleic acid is associated with a virus that causes latent infection. Latent viruses may be, for example, human immunodeficiency virus, human T-cell leukemia virus, Epstein-Barr virus, human cytomegalovirus, human herpesviruses 6 and 7, herpes simplex virus types 1 and 2, varicella-zoster virus, measles virus, or human papilloma viruses. Aspects of the invention allow for fusion polypeptides to be designed to target any virus, latent or active.

Methods of the invention may be used to treat a virus in a mammal by delivering a nucleic acid that encodes a cleaving polypeptide and a sequence-specific targeting polypeptide that targets the cleaving polypeptide to viral nucleic acid thereby causing the cleaving polypeptide to cleave the viral nucleic acid.

### BRIEF DESCRIPTION OF THE DRAWINGS

FIG. 1 shows a first composition for treating a viral infection.

FIG. 2 shows a map of an EBV genome.

FIG. 3 diagrams a method of the invention.

FIG. 4 shows a second composition for treating a viral infection.

FIG. 5 shows a sequence from the HPV 18 viral genome along with various HPV 18 TALENs designed to bind multiple E6 gene segments.

FIG. 6 shows targeted regions of the HPV 18 E6 gene.

FIG. 7 shows viable cell counts for HPV 18+ HeLa cells transfected with plasmid DNA encoding certain TALEN and CRISPR/Cas9 complexes 5 days after transfection.

### DETAILED DESCRIPTION

The invention generally relates to compositions and methods for selectively treating viral infections using a targeting peptide linked to a cleaving peptide, wherein the peptides can be polypeptides. Compositions and methods of the invention are used to incapacitate or disrupt viral nucleic acid within a cell through nuclease activity such as single- or double-stranded breaks, cleavage, digestion, or editing. Composition and methods of the invention are also used for systematically causing large or repeated deletions in the genome, reducing the probability of reconstructing the full genome.

i. Targeting Polypeptide

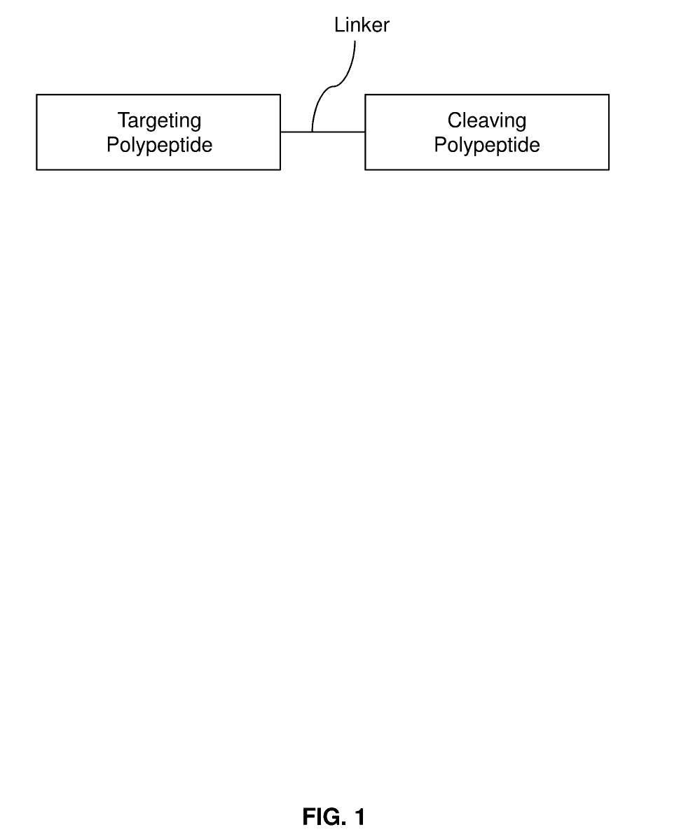

Compositions and methods of the invention include the use of a targeting polypeptide that binds specifically to a specific viral nucleic acid and that is linked to a cleaving polypeptide that cleaves viral nucleic acid (see, e.g., FIG. 1). The composition comprising the targeting polypeptide and the cleaving polypeptide can be a fusion polypeptide, wherein the term fusion polypeptide is meant herein to encompass all manners for linking the two polypeptides. The targeting polypeptide functions to lead the fusion polypeptide to the viral nucleic acid in order to cause genomic disruption. The targeting polypeptide can be chosen to target specific viruses within a cell. The nucleic acid of any virus may be targeted by the targeting polypeptide for cleavage by the cleaving polypeptide. Examples of various viruses, the nucleic acid of which is to be targeted by the targeting polypeptide, include but are not limited to, herpes simplex virus (HSV)-1, HSV-2, varicella zoster virus (VZV), Epstein-Barr virus (EBV), cytomegalovirus (CMV), human herpesvirus (HHV)-6A and -6B, HHV-7, Kaposi's sarcoma-associated herpesvirus (KSHV), JC virus, BK virus, parvovirus b19, adeno-associated virus (AAV), adenovirus, Human papillomavirus (HPV), JC virus, Smallpox, Hepatitis B virus, Human bocavirus, Human astrovirus, Norwalk virus, coxsackievirus, hepatitis A virus, poliovirus, rhinovirus, severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus (RSV), Human metapneumovirus, Hendra virus, Nipah virus, Rabies virus, Hepatitis D, Rotavirus, Orbivirus, Coltivirus, and Banna virus. In one embodiment, the virus is a member of the herpesviridae family, e.g., herpes simplex virus (HSV)-1, HSV-2, varicella zoster virus (VZV), Epstein-Barr virus (EBV), cytomegalovirus (CMV), human herpesvirus (HHV)-6A and -6B, HHV-7, and Kaposi's sarcoma-associated herpesvirus (KSHV). In one aspect of the embodiment, the virus is the Epstein-Barr virus. In another embodiment, the virus is HPV.

Suitable targeting polypeptides for targeting and binding to viral nucleic acid can include, but are not limited to various proteins that bind to viral nucleic acid in a sequence specific manner, or “binding proteins,” such as viral proteins, zinc-finger proteins, transcription activator-like effector (TALE) proteins, the binding moiety of clustered regularly interspaced short palindromic repeat (CRISPR)/Cas guide RNAs and meganucleases. See Schiffer, 2012, Targeted DNA mutagenesis for the cure of chronic viral infections, J Virol 88(17):8920-8936, incorporated by reference.

The targeting polypeptide can be a naturally occurring protein that binds to viral nucleic acid. The targeting polypeptide can also be a non-natural, or genetically engineered, polypeptide that matches a sequence in a naturally occurring protein that binds to viral nucleic acid by at least 95 percent. In some aspects, the polypeptide matches the sequence in a naturally occurring protein by at least 96, 97, 98, 99 or 100%.

In one embodiment, the targeting polypeptide is a viral protein that binds to viral nucleic acid in a sequence specific manner, or a “viral binding protein.” Exemplary viral binding proteins include herpes simplex virus protein vmw65, EBNA-1, EBNA-2, EBNA-3, LMP-1, LMP-2 and EBER from EBV and E1 and E2 from HPV.

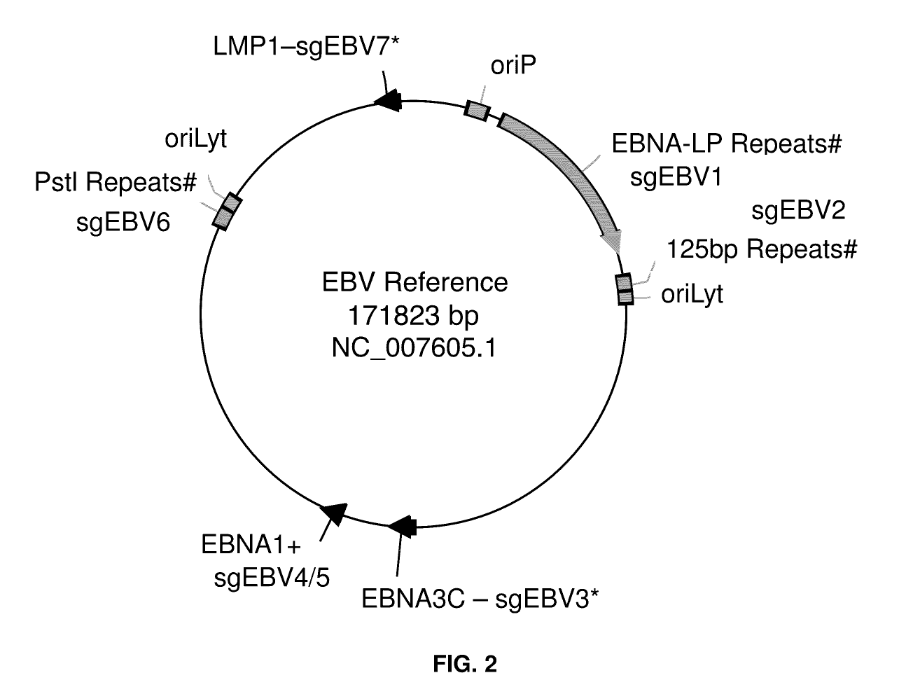

In one embodiment, the targeting polypeptide is predisposed to target viral nucleic acid of a latent virus. For example, the targeting polypeptide can be a viral binding protein that is coded for the latent virus to be targeted. As noted above, EBNA1 is an example of a viral protein that is coded for a latent virus to be targeted. EBNA1 is the only nuclear EBV protein expressed in both latent and lytic modes of infection and is integral in many EBV functions including gene regulation, extrachromosomal replication, and maintenance of the EBV episomal genome through positive and negative regulation of viral promoters. See, e.g., Duellman et al., 2009, “Phosphorylation sites of Epstein-Barr Virus EBNA1 regulate its function”, J Gen Virol. 90 (9): 2251-9 and Kennedy & Sugden, 2003, “EBNA1, a Bifunctional Transcription Activator”, Molecular and Cellular Biology 23 (19): 6901-6908, each incorporated by reference. Studies show that the phosphorylation of ten specific sites on EBNA1 regulates these functions. When phosphorylation does not occur, replication and transcription activities of the protein are significantly decreased. See Duellman (2009). EBNA1 acts through sequence-specific binding to the plasmid origin of viral replication (oriP) within the viral episome. The oriP has four EBNA1 binding sites where replication is initiated as well as a 20-site repeat segment which also enhances the presence of the protein. See, e.g. Young & Murry, 2003, “Epstein-Barr Virus and oncogenesis: from latent genes to tumors”, Oncogene 22(33):5108-5121. EBNA1's binding specificity, as well as its ability to tether EBV DNA to chromosomal DNA, allows EBNA1 to mediate replication and partitioning of the episome during division of the host cell. See, e.g., Young & Rickinson, 2004, “Epstein-Barr Virus: 40 Years On”, Nature Reviews—Cancer 4 (10):757-68 and Levitskaya J, Coram M, Levitsky et al., 1995, “Inhibition of antigen processing by the internal repeat region of the Epstein-Barr virus nuclear antigen-1”, Nature 375(6533):685-8, each incorporated by reference. EBNA1 also interacts with some viral promoters via several mechanisms, further contributing to transcriptional regulation of EBNA1 itself as well as the other EBNAs (2 and 3) and of EBV latent membrane protein 1 (LMP1). See, e.g., Young (2004). Thus, as can be seen in FIG. 2, the EBV genome will be cleaved within the OriP by compositions of the invention.

A fusion polypeptide comprising EBNA1 will target and bind to its coding region within the viral genome. The linked cleaving polypeptide can then cleave the viral genome at either or both ends of the targeted coding region such that the region is excised. These targets enable systematic digestion of the EBV genome into smaller pieces, which will render EBV incapacitated.

In another embodiment, the targeting polypeptide can be a modified CRISPER/Cas system that utilizes a catalytically dead Cas9 (dCas9). Clustered Regularly Interspaced Short Palindromic Repeats (CRISPR) is found in bacteria and is believed to protect the bacteria from phage infection. The gene sequence of a CRISPER/Cas system is made up of the CRISPER locus, which encodes RNA components of the system and the Cas (CRISPR associated) locus, which encodes proteins. The targeting polypeptide may be a catalytically inactive version of high-fidelity Cas9 (hi-fi Cas9), which is described in Kleinstiver et al., 2016, High-fidelity CRISPR-Cas9 nucleases with no detectable genome-wide off-target effects, Nature 529:490-495, incorporated by reference.

In CRISPR systems, Cas complexes with small RNAs as guides (gRNAs) to target and cleave DNA in a sequence-specific manner. In a CRISPR system comprising dCas9, the Cas9 nuclease has been catalytically inactived, such that the CRISPER/Cas complex will no longer cleave DNA. See, e.g., Maeder et al., “CRISPR RNA-guided activation of endogenous human genes.” Nat. Methods (October 2013), 10(10):977-979. This is accomplished by introducing point mutations in the two catalytic residues (D10A and H480A) of the gene encoding Cas9. Jinek et al., (2012) “A Programmable Dual-RNA-Guided DNA Endonuclease in Adaptive Bacterial Immunity.” Science 337 (6096): 816-821. Separate guide RNAs, known as the crRNA and tracrRNA, may be used. These two separate RNAs have been combined into a single RNA to enable site-specific mammalian genome targeting through the design of a short guide RNA. The dCas9 and guide RNA (gRNA) may be synthesized by known methods. FIG. 2 shows a map of the EBV genome with various guide RNAs (e.g., sgEBV1-7) and their targets indicated. In one embodiment, a composition according to the invention includes a dCas9-gRNA complex linked to a cleaving polypeptide. In another embodiment, the composition includes genes encoding for the dCas9-gRNA complex, linker and cleaving polypeptide. In one aspect of the embodiments, the cleaving polypeptide is the cleavage domain of FokI.

In other embodiments, the fusion polypeptides can include a TALE (transcription activator-like effector) DNA binding domain. See U.S. Pat. No. 8,586,526. TALE are proteins secreted by Xanthomonas bacteria via their type III secretion system when they infect various plant species. The DNA-binding domain can be naturally occurring or can be engineered to target essentially any sequence. For TALE technology, target sites are identified and expression vectors are made. The DNA binding domain contains a repeated highly conserved 33-34 amino acid sequence with the exception of the 12th and 13th amino acids. These two locations are highly variable (Repeat Variable Diresidue) and show a strong correlation with specific nucleotide recognition. See, e.g. Boch, Jens et al. (December 2009). “Breaking the Code of DNA Binding Specificity of TAL-Type III Effectors”. Science 326 (5959): 1509-12; and Moscou, Matthew J.; Adam J. Bogdanove (December 2009). “A Simple Cipher Governs DNA Recognition by TAL Effectors”. Science 326 (5959): 1501. By selecting a combination of repeat segments containing the appropriate RVDs, the relationship between amino acid sequence and DNA recognition enables the engineering of specific DNA-binding domains. See, e.g., Boch, Jens (February 2011). “TALEs of genome targeting”. Nature Biotechnology 29 (2): 135-6. Linearized expression vectors (e.g., by NotI) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55.

TALEs and CRISPR methods provide one-to-one relationship to the target sites, i.e. one unit of the tandem repeat in the TALE domain recognizes one nucleotide in the target site, and the crRNA, gRNA, or sgRNA of a CRISPR/dCas9 system hybridizes to the complementary sequence in the DNA target. Methods can include using a pair of TALEs or a dCas9 protein with one gRNA to target the DNA for cleavage by the cleaving polypeptide. The breaks can optionally be repaired via non-homologous end-joining (NHEJ) or homologous recombination (HR).

In yet another embodiment, the targeting polypeptide can be a zinc finger protein. Zinc finger binding domains may be engineered to recognize and bind to any nucleic acid sequence of choice. See, e.g., Qu et al., 2013, Zinc-finger-nucleases mediate specific and efficient excision of HIV-1 proviral DNA from infected and latently infected human T cells, Nucl Ac Res 41(16):7771-7782; and Beerli et al., 2002, Engineering polydactyl zinc-finger transcription factors, Nature Biotechnol 20:135-141, incorporated by reference. The specificity of an engineered protein is preferred, compared to a naturally occurring zinc finger. Methods for engineering zinc finger proteins include, but are not limited to, rational design and various selection methods. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference. In one embodiment, a composition according to the invention includes a zinc finger protein linked to a cleaving polypeptide. In another embodiment, the composition includes genes encoding for the zinc finger protein, linker and cleaving polypeptide. In one aspect of the embodiments, the cleaving polypeptide is the cleavage domain of FokI.

In another embodiment the targeting polypeptide can be the binding domain of a meganuclease. Meganucleases (homing endonuclease) are endo-deoxyribonucleases characterized by a large recognition site (double-stranded DNA sequences of 12 to 40 base pairs). As a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs. The most well studied family has been found in all kingdoms of life, generally encoded within introns or inteins although freestanding members also exist. They contain sequence motif that represents an essential element for enzymatic activity. Some proteins contained only one such motif, while others contained two; in both cases the motifs were followed by ˜75-200 amino acid residues having little to no sequence similarity with other family members. Crystal structures illustrates mode of sequence specificity for the meganucleases: specificity contacts arise from the burial of extended β-strands into the major groove of the DNA, with the DNA binding saddle having a pitch and contour mimicking the helical twist of the DNA; full hydrogen bonding potential between the protein and DNA is never fully realized; (and additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference. Additionally, the DNA-binding specificity of meganucleases can be engineered to bind non-natural target sites.

In one embodiment, a composition according to the invention includes the binding domain of a meganuclease linked to a cleaving polypeptide. In another embodiment, the composition includes genes encoding for the binding domain of a meganuclease, linker and cleaving polypeptide. In one aspect of the embodiments, the cleaving polypeptide is the cleavage domain of FokI.

Any suitable catalytically inactive nuclease may be used as a targeting peptide. A targeting peptide may be a catalytically inactive Cas9 homolog or another CRISPR-associated nuclease, ngAgo, Cpf1, or hi-fi Cas9 that has been catalytically inactivated. The targeting peptide may be for example, a catalytically inactive version of Cas9, ZFNs, TALENs, Cpf1, NgAgo, or a modified programmable nuclease having an amino acid sequence substantially similar to the unmodified version, for example, a programmable nuclease having an amino acid sequence at least 85% similar to one of Cas9, ZFNs, TALENs, Cpf1, or NgAgo, or any other programmable nuclease. The targeting peptide may be provided by a catalytically inactive programmable nuclease. Programmable nuclease generally refers to an enzyme that cleaves nucleic acid that can be or has been designed or engineered by human contribution so that the enzyme targets or cleaves the nucleic acid in a sequence-specific manner.

ii. Cleaving Polypeptide

Methods of the invention include using a composition such as a fusion polypeptide that includes a cleaving polypeptide linked to the targeting polypeptide, the targeting polypeptide specifically targeting and binding to viral nucleic acid for destruction by the cleaving portion. The cleaving polypeptide can be any suitable endo- or exo-nuclease, including, for example, restriction endonucleases, meganucleases (homing endonucleases), zinc finger nucleases (ZFN), TALEN, and Cas9 nucleases, most of which were described with respect to the targeting polypeptide. A nuclease is an enzyme capable of cleaving the phosphodiester bonds between the nucleotide subunits of nucleic acids. Nucleases are typically divided into one of two categories: endonucleases and exonucleases. Exonucleases cleave nucleotides one at a time from the end (exo) of a polynucleotide chain, while endonucleases cleave the phosphodiester bond within a polynucleotide chain. Some nucleases cut DNA relatively nonspecifically (without regard to sequence), while many, typically called restriction endonucleases or restriction enzymes, cleave only at very specific nucleotide sequences.

In one embodiment, the cleaving polypeptide is the cleavage domain of a nuclease. The term “cleavage domain” also includes “cleavage half-domains”. A cleavage half domain will require dimerization for cleavage activity. In one embodiment, when the fusion polypeptide comprises a cleavage half-domain, two fusion polypeptides may be used to effect cleavage. In another embodiment, when the fusion polypeptide comprises a cleavage half-domain, a single fusion product can comprise two cleavage half-domains. The two cleavage half-domains can be derived from the same nuclease or from a different nuclease.

The nuclease from which the cleavage domain is derived can be any endonuclease or exonuclease. For example, and not to be limiting, cleavage domains can be derived from restriction endonucleases and homing endonucleases. See, for example, 2002-2003 Catalogue, New England Biolabs, Beverly, Mass.; and Belfort et al. (1997) Nucleic Acids Res. 25:3379-3388. Additionally, the following enzymes which cleave DNA are examples of sources of cleavage domains, SI Nuclease; mung bean nuclease; pancreatic DNase I; micrococcal nuclease; and yeast HO endonuclease. See also Linn et al. (eds.) Nucleases, Cold Spring Harbor Laboratory Press, 1993. It is to be appreciated that one or more of these nucleases can be used as a source of cleavage domains.

In one embodiment the cleavage domain can be derived from a restriction endonuclease (restriction enzyme). As noted above, restriction enzymes are capable of binding to DNA in a sequence specific manner (at a recognition site) and cleaving DNA at or near the site of binding. Some restriction enzymes have separable binding and cleavage domains and cleave DNA at sites removed from the recognition site, such that when the domains are separated, the cleavage domain cleaves nucleic acid in a non-sequence specific manner. Such enzymes include, but are not limited to, Type IIS enzymes. Suitable Type IIS enzymes include, but are not limited to, for example, Aar 1, BsrB I, SspD5 I, Ace III, BsrD I, Sth132 I, Aci I, BstF5 I, Sts I, AIo I, Btr I, TspDT I, Bae I, Bts I, TspGW I, Bbr7 I Cdi I Tth1 11 II, Bbv I, CjeP I, UbaP I, Bbv II, Drd II, Bsa I, BbvC I, Eci I, BsmB I, Bed Eco31, Bce83 I, Eco57 I, BceAI, Eco57M I, Bcef I Esp3I, Beg I, Faul, BciVI, Fin I, BfiI, FokI, Bin I, GdiII, BmgI, GsuI, Bpul0I, HgaI, BsaXI, Hin4 II, BsbI, HphI, BscAI, Ksp632 I, BscGI, Mbo π, BseRI, MIyI, BseYI, MmeI, BsiI, MnII, BsmI, PfII, 108 I, BsmAI, PIeI, BsmFI, PpiI, Bsp241, PsrI, BspGI, R1eAI, BspMI, Sap I, BspNC I, SfaNI, Bsr I, and Sim I. Thus, in one embodiment, the fusion protein comprises a cleavage domain derived from at least one Type IIS restriction enzyme. In one aspect of the embodiment, the Type IIS restriction enzyme is the FokI enzyme. Additional restriction enzymes that contain separate binding and cleavage domains are also contemplated.

The FokI enzyme catalyzes double strand cleavage of DNA at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J Biol. Chem. 269:31,978-31,982. As noted above, the FokI enzyme has a cleavage domain that is separable from its binding domain. Additionally, the FokI enzyme is active as a dimer. Bitinaite et al. (1998) Proc. Natl. Acad. Sci. USA 95: 10,570-10,575. As such, dimerization with another FokI cleavage domain is needed to effect cleavage. See Wah, et al., 1998, Structure of FokI has implications for DNA cleavage, PNAS 95:10564-10569. It in envisioned that two fusion proteins, each containing a FokI cleavage domain can be used to cleave targeted nucleic acid. It is also envisioned that the fusion protein can comprise two FokI cleavage domains. See U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference. Because dimerization is needed to activate the FokI enzyme, such that two cleavage domains must be present, the cleavage domain of FokI is often referred to as a half domain. It is also appreciated that the FokI cleavage domain may be modified in any way, such as by additions, deletions and/or substitutions of amino acids.

In one aspect of the invention, the cleaving polypeptide causes a double strand break in at least two locations in the genome of the target virus. These two double strand breaks cause a fragment of the genome to be deleted. Even if viral repair pathways anneal the two ends, there will still be a deletion in the genome. One or more deletions using the fusion polypeptide can incapacitate the viral genome. In an aspect of the invention, the number of deletions lowers the probability that the genome may be repaired. In a highly-preferred embodiment, the fusion polypeptide of the invention causes significant genomic disruption, resulting in effective destruction of the viral genome, while leaving the host genome intact (because the targeting polypeptide binds specifically to viral nucleic acid meaning that it does not bind to human nucleic acid). The desired result is that the host cell will be free of viral infection.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the non-homologous end joining (NHEJ) repair pathway can be leveraged to disrupt the target gene. In a preferred embodiment, numerous insertions are caused in the genome, thereby incapacitating the virus. In an aspect of the invention, the number of insertions lowers the probability that the genome may be repaired.

In some embodiments of the invention, a template sequence can be inserted into the genome. In order to introduce nucleotide modifications to genomic DNA, a DNA repair template containing the desired sequence must be present during homology directed repair (HDR). The DNA template is normally transfected into the cell along with the fusion polypeptide or the vector encoding it. The length and binding position of each homology arm is dependent on the size of the change being introduced. In the presence of a suitable template, HDR can introduce significant changes at the fusion polypeptide-induced double strand break.

Some embodiments of the invention may utilize a modified version of a nuclease. For instance, the nuclease can be modified such that one catalytic domain is inactive. A catalytic domain can be inactivated, for example, by the introduction of a mutation. This type of modified nuclease is referred to as a nickase and cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a nickase are treated as a double strand break, in what is often referred to as a ‘double nick’ or ‘dual nickase’ system. A double-nick induced double strain break can be repaired by either NHEJ or HDR depending on the desired effect on the gene target. At these double strain breaks, insertions and deletions are caused by the fusion polypeptide. In an aspect of the invention, a deletion is caused by positioning two double strand breaks proximate to one another, thereby causing a fragment of the genome to be deleted.

iii. Linkage

A composition of the invention comprises a cleaving polypeptide linked to a targeting polypeptide (or a nucleic acid encoding such features). The cleaving polypeptide can be linked to the targeting polypeptide by way of one or more covalent bonds or by other means. In one embodiment, the at least one covalent bond is a peptide bond. In one aspect of the embodiment, the peptide bond is used to link the cleaving polypeptide to the targeting polypeptide into a polypeptide sequence. The sequence joining the cleaving polypeptide and the targeting polypeptide can comprise one or more amino acids in any sequence that does not substantially hinder the ability of the targeting polypeptide to bind to its target site or the cleavage domain to cleave the viral nucleic acid.

In some embodiments, the cleaving portion is linked to the targeting portion by bonds that involve atoms of the amino acid side chains. For example, one or more disulfide bonds involving cysteine residues in the polypeptides.

Additionally, the composition can comprise a cleaving polypeptide linked to a targeting polypeptide by way of a biotin/streptadivin linkage. The binding of biotin to streptavidin is one of the strongest non-covalent interactions known. The most common use of biotin-streptadivin linkages are for the purification and detection of various biomolecules, but has also found use in the creation of nanoscale devices and structures. See, e.g., Holmberg, Anders; Blomstergren, Anna; Nord, Olof; Lukacs, Morten; Lundeberg, Joakim; Uhlén, Mathias (2005). “The biotin-streptavidin interaction can be reversibly broken using water at elevated temperatures”. Electrophoresis 26 (3): 501-10; and Osojic, G N; Hersam, M C (2012). “Biomolecule-Directed Assembly of Self-Supported, Nanoporous, Conductive, and Luminescent Single-Walled Carbon Nanotube Scaffolds”. Small 8 (12): 1840-5.

iv. Delivery

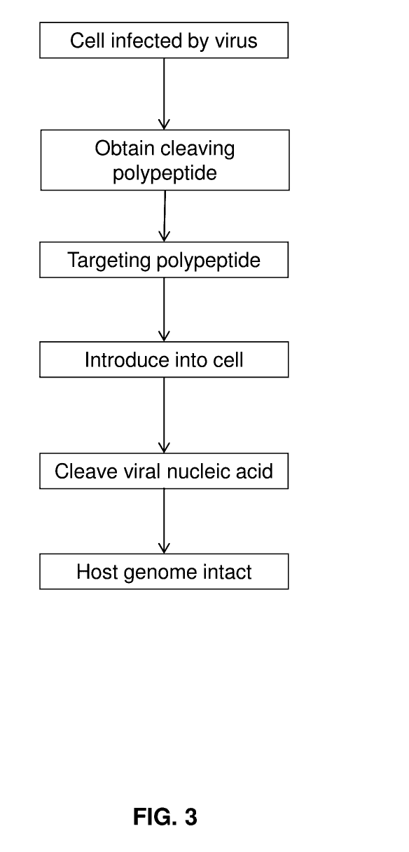

FIG. 3 diagrams a method of treating a cell infected with a virus. Methods of the invention are applicable to in vivo treatment of patients and may be used to remove any viral genetic material such as genes of virus associated with a latent viral infection. Methods may be used in vitro, e.g., to prepare or treat a cell culture or cell sample. When used in vivo, the cell may be any suitable germ line or somatic cell and compositions of the invention may be delivered to specific parts of a patient's body or be delivered systemically. If delivered systemically, it may be preferable to include within compositions of the invention tissue-specific promoters. For example, if a patient has a latent viral infection that is localized to the liver, hepatic tissue-specific promotors may be included in a plasmid or viral vector that codes for a targeted nuclease.

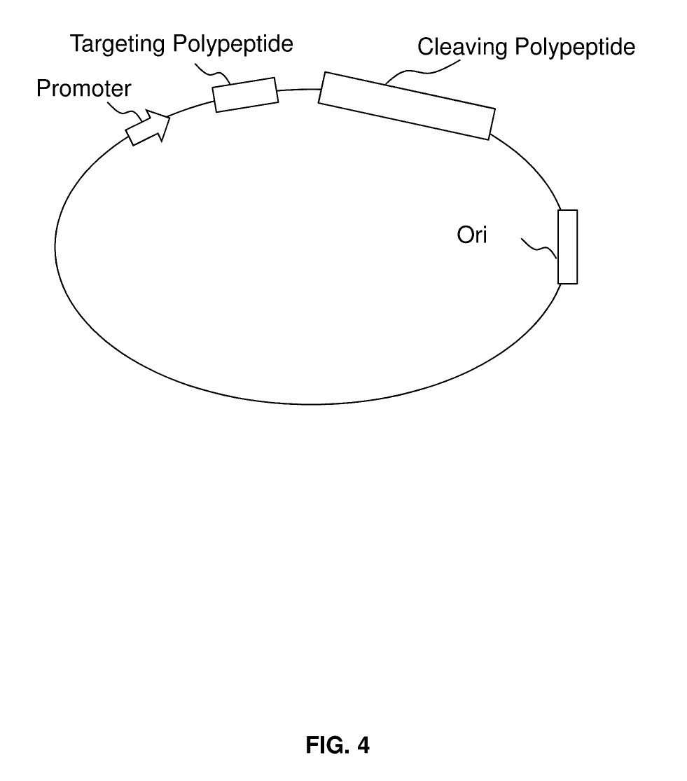

FIG. 4 shows a composition for treating a viral infection according to certain embodiments. The composition preferably includes a vector (which can be, for example, a plasmid, linear DNA, or a viral vector) that codes for a cleaving polypeptide and a targeting polypeptide that targets the cleaving polypeptide to viral nucleic acid. The composition may optionally include one or more of a promoter, replication origin, gene encoding a nuclear localization signal (NLS), other elements, or combinations thereof as described further herein.

Methods of the invention include introducing into a cell a composition, such as a fusion polypeptide, comprising a cleaving polypeptide and a sequence-specific targeting polypeptide. Any suitable method can be used to deliver, for example, the fusion polypeptide to the infected cell or tissue. For example, but not to be limited by, the fusion polypeptide or the nucleic acid encoding the fusion polypeptide may be delivered topically, by injection, orally, or by hydrodynamic delivery. The fusion polypeptide or the nucleic acid encoding the fusion polypeptide may be delivered to systematic circulation or may be delivered or otherwise localized to a specific tissue type. The fusion polypeptide or the nucleic acid encoding the fusion polypeptide may be modified or programmed to be active under only certain conditions such as by using a tissue-specific promoter so that the encoded fusion polypeptide is preferentially or only transcribed in certain tissue types.

In some embodiments, a fusion polypeptide comprising a protein that binds to viral nucleic acid (“binding protein”) and the cleavage domain of a nuclease are introduced into a cell. As noted previously, one example of a binding protein in accordance with the invention is a protein that binds viral nucleic acid. Such a protein by its nature is targeted to a specific sequence of the viral genome. In addition to latent infections, this invention can also be used to control actively replicating viruses by targeting the viral genome before it is packaged or after it is ejected.

In some embodiments, a cocktail of binding proteins may be introduced into a cell. The proteins can target numerous categories of sequences of a viral genome. By targeting several areas along the genome, the double strand breaks at multiple locations fragment the genome, lowering the possibility of repair. Even with repair mechanisms, the large deletions render the virus incapacitated. For example, two to twelve targeting polypeptides may be used. In another embodiment, one, two, three, four, five, six, seven, eight, nine, ten, eleven or twelve targeting polypeptides may be used, which target different categories of sequences. However, any number of targeting polypeptides may be introduced into a cocktail to target categories of sequences. In preferred embodiments, the categories of sequences are important for genome structure, host cell transformation, and infection latency, respectively. In one embodiment, one or more of the latent EBV proteins, EBNA-1, EBNA-2, EBNA-3, LMP-1, LMP-2 and EBER are introduced into the cell. It is also to be understood that this disclosure extends to any cleaving polypeptide of the invention.

In some aspects of the invention, in vitro experiments allow for the determination of the most essential targets within a viral genome. For example, to understand the most essential targets for effective incapacitation of a genome, subsets of targeting moieties can be transfected into model cells. Assays can determine which targeting moieties or which cocktail is the most effective at targeting essential categories of sequences.

For example, in the case of the EBV genome targeting, the latent proteins include the six nuclear antigens (EBNAs 1, 2, 3A, 3B and 3C, and EBNA-LP) and the three latent membrane proteins (LMPs 1, 2A and 2B). Therefore, assays could be developed to determine which protein or combinations of proteins were most effective at incapacitating the EBV genome.

Once the fusion polypeptides are constructed, the compositions, or genes encoding the compositions, can be introduced into a cell. It should be appreciated that the compositions can be introduced into cells in an in vitro model or an in vivo model. The compositions of the invention can be transfected into cells by various methods. In one aspect, genes encoding the fusion polypeptide can be introduced into cells by vectors. Suitable vectors include viral vectors and non-viral vectors. Examples of suitable viral vectors include, but are not limited to, retroviruses, lentiviruses, adenoviruses, and adeno-associated viruses. It should be appreciated that any viral vector may be incorporated into the present invention to effectuate delivery of genes encoding the fusion polypeptide into a cell. Some viral vectors may be more effective than others, depending on the fusion polypeptide designed for digestion or incapacitation. In an aspect of the invention, the vectors contain essential components such as origin of replication, which is necessary for the replication and maintenance of the vector in the host cell. Use of viral vectors as delivery vectors are known in the art. See for example U.S. Pub. 2009/0017543 to Wilkes et al., the contents of which are incorporated by reference.

A retrovirus is a single-stranded RNA virus that stores its nucleic acid in the form of an mRNA genome (including the 5′ cap and 3′ PolyA tail) and targets a host cell as an obligate parasite. In some methods in the art, retroviruses have been used to introduce nucleic acids into a cell. Once inside the host cell cytoplasm the virus uses its own reverse transcriptase enzyme to produce DNA from its RNA genome, the reverse of the usual pattern, thus retro (backwards). This new DNA is then incorporated into the host cell genome by an integrase enzyme, at which point the retroviral DNA is referred to as a provirus. For example, the recombinant retroviruses such as the Moloney murine leukemia virus have the ability to integrate into the host genome in a stable fashion. They contain a reverse transcriptase that allows integration into the host genome. Retroviral vectors can either be replication-competent or replication-defective. In some embodiments of the invention, retroviruses are incorporated to effectuate transfection into a cell,

Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides. In some embodiments of the invention, lentiviruses are used as viral vectors.

As opposed to lentiviruses, adenoviral DNA does not integrate into the genome of the host and is not replicated during cell division. A related virus, adeno-associated virus (AAV), is a small virus that infects humans and some other primate species. While the native AAV can incorporate its genome into that of a host cell, it persist in a state that does not integrate into the genome of a host when used a vector. Therefore adenoviruses and the adeno-associated viruses (AAV) are potential approaches as delivery vectors when integration into the host's genome is not desired. In some aspects of the invention, only the viral genome to be targeted is effected by the fusion protein, and not the host's cells. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Additional viral vectors may also include, but are not limited to herpesvirus, poxvirus, alphavirus, or vaccinia virus.

In certain embodiments of the invention, non-viral vectors may be used to effectuate transfection. Suitable non-viral vectors and methods of delivering non-viral vectors include, but are not limited to, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Because degradation of nucleic acid can occur, several methods for protecting nucleic acid are available. In one embodiment, synthetic vectors, which are typically based on cationic lipids or polymers, can be used. Synthetic vectors can complex with negatively charged nucleic acids to form particles with a diameter in the order of 100 nm. The complex protects nucleic acid from degradation by nuclease.

Additionally, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. As such, methods for mitigating these adverse events are available. For example, modifying the surfaces of cationic non-viral vectors can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor. In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

However, PEG on the surface can decrease the uptake by target cells and reduce the biological activity. Therefore, attaching a targeting ligand to the distal end of the PEGylated component is necessary. For example, the ligand is projected beyond the PEG “shield” to allow binding to receptors on the target cell surface. When cationic liposome is used as gene carrier, the application of neutral helper lipid is helpful for the release of nucleic acid, besides promoting hexagonal phase formation to enable endosomal escape. In some embodiments of the invention, neutral or anionic liposomes are developed for systemic delivery of nucleic acids and obtaining therapeutic effect in experimental animal model. Designing and synthesizing novel cationic lipids and polymers, and covalently or noncovalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called ‘photothermal effect’. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome.

In some embodiments, the complexes of the invention are encapsulated in a liposome. Unlike small molecule drugs, nucleic acids cannot cross intact lipid bilayers, predominantly due to the large size and hydrophilic nature of the nucleic acid. Therefore, nucleic acids may be entrapped within liposomes with conventional passive loading technologies, such as ethanol drop method (as in SALP), reverse-phase evaporation method, and ethanol dilution method (as in SNALP).

In some embodiments, linear polyethylenimine (L-PEI) is used as a non-viral vector due to its versatility and comparatively high transfection efficiency. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

Low-intensity ultrasound in combination with microbubbles has recently acquired much attention as a safe method of gene delivery. Ultrasound shows tissue-permeabilizing effect. It is non-invasive and site-specific, and could make it possible to destroy tumor cells after systemic delivery, while leave nontargeted organs unaffected. Ultrasound-mediated microbubbles destruction has been proposed as an innovative method for noninvasive delivering of drugs and nucleic acids to different tissues. Microbubbles are used to carry a drug or gene until a specific area of interest is reached, and then ultrasound is used to burst the microbubbles, causing site-specific delivery of the bioactive materials. Furthermore, the ability of albumin-coated microbubbles to adhere to vascular regions with glycocalix damage or endothelial dysfunction is another possible mechanism to deliver drugs even in the absence of ultrasound. See Tsutsui et al., 2004, “The use of microbubbles to target drug delivery,” Cardiovasc Ultrasound 2:23, the contents of which are incorporated by reference. In ultrasound-triggered drug delivery, tissue-permeabilizing effect can be potentiated using ultrasound contrast agents, gas-filled microbubbles. The use of microbubbles for delivery of nucleic acids is based on the hypothesis that destruction of DNA-loaded microbubbles by a focused ultrasound beam during their microvascular transit through the target area will result in localized transduction upon disruption of the microbubble shell while sparing non-targeted areas.

Besides ultrasound-mediated delivery, magnetic targeting delivery could be used for delivery. Magnetic nanoparticles are usually entrapped in gene vectors for imaging the delivery of nucleic acid. Nucleic acid carriers can be responsive to both ultrasound and magnetic fields, i.e., magnetic and acoustically active lipospheres (MAALs). The basic premise is that therapeutic agents are attached to, or encapsulated within, a magnetic micro- or nanoparticle. These particles may have magnetic cores with a polymer or metal coating which can be functionalized, or may consist of porous polymers that contain magnetic nanoparticles precipitated within the pores. By functionalizing the polymer or metal coating it is possible to attach, for example, cytotoxic drugs for targeted chemotherapy or therapeutic DNA to correct a genetic defect. Once attached, the particle/therapeutic agent complex is injected into the bloodstream, often using a catheter to position the injection site near the target. Magnetic fields, generally from high-field, high-gradient, rare earth magnets are focused over the target site and the forces on the particles as they enter the field allow them to be captured and extravasated (evicted from the blood stream and into the neighboring tissue) at the target.

Synthetic cationic polymer-based nanoparticles (˜100 nm diameter) have been developed that offer enhanced transfection efficiency combined with reduced cytotoxicity, as compared to traditional liposomes. The incorporation of distinct layers composed of lipid molecules with varying physical and chemical characteristics into the polymer nanoparticle formulation resulted in improved efficiency through better fusion with cell membrane and entry into the cell, enhanced release of molecules inside the cell, and reduced intracellular degradation of nanoparticle complexes.

In some embodiments, the complexes are conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70.

Liposomal delivery systems provide stable formulations, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

Some embodiments of the invention provide for a gene gun or a biolistic particle delivery system. A gene gun is a device for injecting cells with genetic information, where the payload may be an elemental particle of a heavy metal coated with plasmid DNA. This technique may also be referred to as bioballistics or biolistics. Gene guns have also been used to deliver DNA vaccines. The gene gun is able to transfect cells with a wide variety of organic and non-organic species, such as DNA plasmids, fluorescent proteins, dyes, etc.

Aspects of the invention provide for numerous uses of delivery vectors. Selection of the delivery vector is based upon the cell or tissue targeted and the specific makeup of the fusion polypeptide.

v. Cut Viral Nucleic Acid

Once inside the cell, the composition targets the viral genome. In addition to latent infections, this invention can also be used to control actively replicating viruses by targeting the viral genome before it is packaged or after it is ejected. In some embodiments, methods and compositions of the invention use a sequence-specific targeting polypeptide such as viral protein to target latent viral genomes, thereby reducing the chances of proliferation. The targeting polypeptide may form a complex with a cleaving polypeptide, such as the cleavage domain of a nuclease. The composition, such as a fusion polypeptide, cuts the viral nucleic acid in a targeted fashion to incapacitate the viral genome. As discussed above, the fusion polypeptide can cause a break in the viral genome. By targeting several locations along the viral genome, the genome is cut at several locations. In one embodiment, double strand breaks are designed so that small deletions are caused or small fragments are removed from the genome so that even if natural repair mechanisms join the genome together, the genome is render incapacitated. Preferably the deleted fragments include 3N±1 nucleotides, where N is a positive integer, to ensure a frameshift thereby shifting any downstream open reading frame out of frame. The fusion polypeptide, or nucleic acid encoding the fusion polypeptide, may be delivered into an infected cell by transfection. For example, the infected cell can be transfected with DNA that encodes EBNA1 and the cleavage domain of FokI (on a single piece or separate pieces).

vi. Host Genome

It will be appreciated that methods and compositions of the invention can be used to target viral nucleic acid without interfering with host genetic material. Methods and compositions of the invention employ a targeting polypeptide that binds specifically to a target within the viral sequence. Methods and compositions of the invention may further use a cleaving polypeptide such as the cleavage domain of FokI, or a vector encoding such polypeptides, which uses the targeting polypeptide to bind exclusively to the viral genome and make double stranded cuts, thereby removing the viral sequence from the host.

For example, where the targeting polypeptide includes a viral nucleic acid binding protein, the sequence is, by its nature, specific to a portion of the viral nucleic acid. Preferably the targeting polypeptide is selected so that the same sequence does not appear in the host genome. Accordingly, viral nucleic acid can be cleaved without interfering with the host genetic material. When other compositions in accordance with the invention are used, it is preferable to choose a sequence such that the composition will bind to and digest specified features or targets in the viral sequence without interfering with the host genome. Where multiple candidate targets are found in the viral genome, selection of the sequence to be the template for the targeting polypeptide may favor the candidate target closest to, or at the 5′ most end of, a targeted feature as the guide sequence. The selection may preferentially favor sequences with neutral (e.g., 40% to 60%) GC content. Additional background with respect to RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes.

Due to the existence of human genomes background in the infected cells, a set of steps are provided to ensure high efficiency against the viral genome and low off-target effect on the human genome. Those steps may include (1) target selection within viral genome, (2) methodologically selecting viral target that is conserved across strains, (3) selecting target with appropriate GC content, (4) control of nuclease expression in cells, (5) vector design, (6) validation assay, others and various combinations thereof. A targeting polypeptide preferably binds to targets within certain categories such as (i) latency related targets, (ii) infection and symptom related targets, and (iii) structure related targets.

With respect to latency related targets, the viral genome requires certain features in order to maintain the latency. These features include, but not limited to, master transcription regulators, latency-specific promoters, signaling proteins communicating with the host cells, etc. If the host cells are dividing during latency, the viral genome requires a replication system to maintain genome copy level. Viral replication origin, terminal repeats, and replication factors binding to the replication origin are great targets. Once the functions of these features are disrupted, the viruses may reactivate, which can be treated by conventional antiviral therapies.

With respect to infection-related and symptom-related targets, a virus produces various molecules to facilitate infection. Once the virus has gained entrance to the host cells, the virus may start a lytic cycle, which can cause cell death and tissue damage. In certain cases, such as with HPV16, cell products (E6 and E7 proteins) can transform the host cells and cause cancers. Disrupting key genome sequences (promoters, coding sequences, etc.) that produce these molecules can prevent further infection, and/or relieve symptoms, if not cure the disease.

With respect to structure-related targets, a viral genome may contain repetitive regions to support genome integration, replication, or other functions. Targeting repetitive regions can break the viral genome into multiple pieces, which physically destroys the genome. It may be preferable to use a targeting polypeptide that targets portions of the viral genome that are highly conserved. Viral genomes are much more variable than human genomes. In order to target different strains, the targeted polypeptide will preferably target conserved regions. As PAM is important to initial sequence recognition, it is also essential to have PAM in the conserved region.

In a preferred embodiment, methods of the invention are used to deliver a nucleic acid to cells. The nucleic acid delivered to the cells may encode a cleaving polypeptide and a targeting polypeptide, or the nucleic acid may include a vector, such as a plasmid, that encodes a cleaving polypeptide and a targeting polypeptide to target and cleave genetic material. Expression of cleaving polypeptide allows it to degrade or otherwise interfere with the target genetic material. The cleaving polypeptide may be a binding protein.

The binding protein targets the cleaving polypeptide to the target genetic material. Where the target genetic material includes the genome of a virus, the binding protein will bind in a sequence specific manner to that genome and can guide the degradation of that genome by the cleaving polypeptide, thereby preventing any further replication or even removing any intact viral genome from the cells entirely. By these means, latent viral infections can be targeted for eradication.

The host cells may grow at different rate, based on the specific cell type. High nuclease expression is necessary for fast replicating cells, whereas low expression help avoiding off-target cutting in non-infected cells. Control of nuclease expression can be achieved through several aspects. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc.) motif into the sequence.

Using the above principles, methods and compositions of the invention may be used to target viral nucleic acid in an infected host without adversely influencing the host genome. Since the targeted locations are selected to be within certain categories such as (i) latency related targets, (ii) infection and symptom related targets, or (iii) structure related targets, cleavage of those sequences inactivates the virus and removes it from the host. Since the fusion polypeptides are designed to match the target in the viral genetic sequence without any off-target matching of the host genome, the latent viral genetic material is removed from the host without any interference with the host genome.

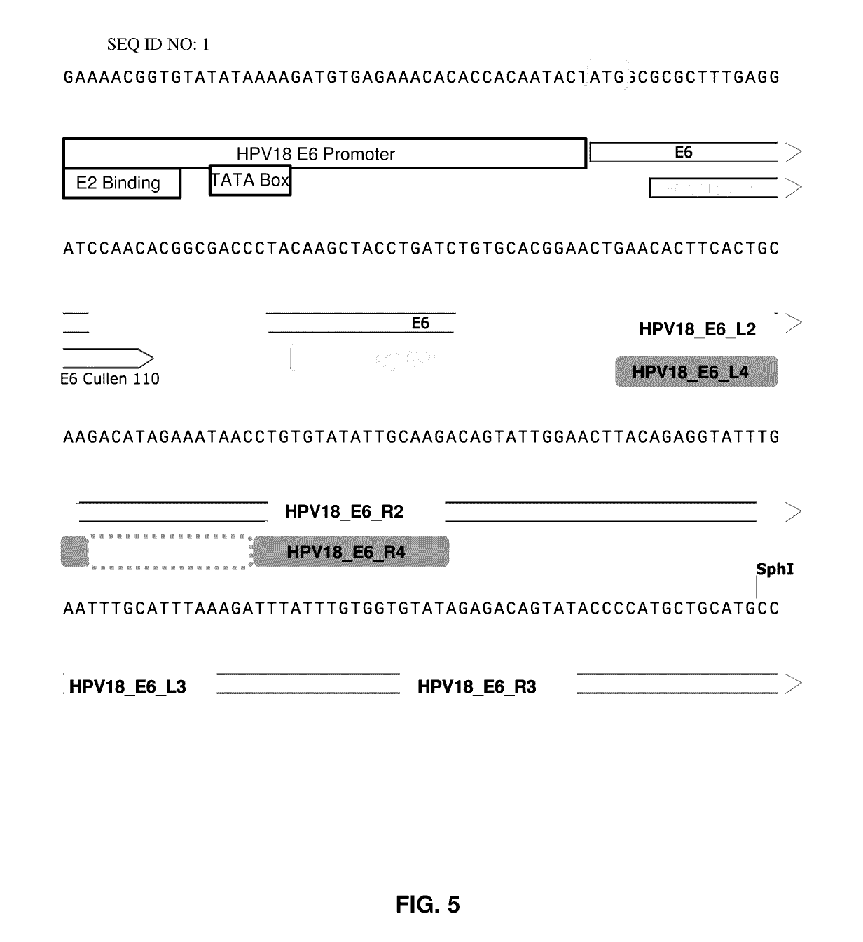

As noted, fusion polypeptides of the invention can include a TALE DNA binding domain. FIG. 5 shows a sequence from the HPV 18 viral genome (SEQ ID NO: 1; GenBank accession number: X05015.1) along with various HPV 18 TALENs designed to bind multiple E6 gene segments. The E6 gene is required for cell transformation and ongoing replication. Pairs of TALENs comprising HPV18_E6_L1 and R1, L2 and R2, L3 and R3, or L4 and R4 are shown. Also illustrated in FIG. 5 is the HPV 18 E6 gene target sequence of a guide RNA (sgE6-2) for use with a guided nuclease such as Cas9 or dCas9.

The depicted portion of the HPV genome is

(SEQ ID NO.: 1)
GAAAACGGTG TATATAAAAG ATGTGAGAAA CACACCACAA
TACTATGGCG CGCTTTGAGG ATCCAACACG GCGACCCTAC
AAGCTACCTG ATCTGTGCAC GGAACTGAAC ACTTCACTGC
AAGACATAGA AATAACCTGT GTATATTGCA AGACAGTATT
GGAACTTACA GAGGTATTTG AATTTGCATT TAAAGATTTA
TTTGTGGTGT ATAGAGACAG TATACCCCAT GCTGCATGCC.

### Example 1: HPV 18-Specific TALENs Shown to Kill HPV 18+ Cancer Cells

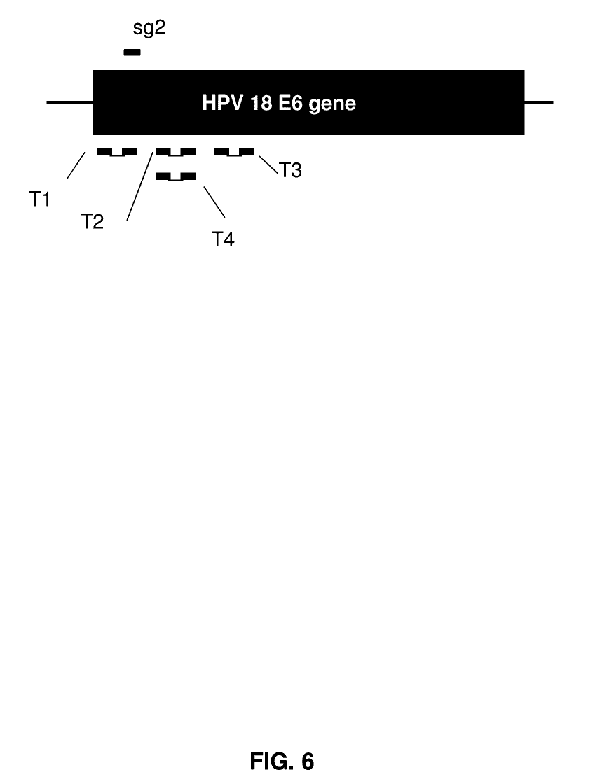

Fusion polypeptides including TALE DNA binding domains may be used to kill HPV 18+ cancer cells. Fusion polypeptides may be expressed in cells that have been transfected with plasmid DNA encoding the fusion polypeptide. HPV 18+ HeLa cells were plated and then transfected the next day with plasmid DNA complexed with cationic liposome. Plasmids encoding various TALENs were used included pAAVS1Talen1, pHPV18E6Talen1 (T1), pHPV18E6Talen2 (T2), pHPV18E6Talen3 (T3), pHPV18E6Talen4 (T4). Plasmids encoding the p113-HPV18E6-2-Cas9 (sg2) and p102-AAVS1-Cas9 complexes were also used. The targeted regions of the HPV 18 E6 gene are shown in FIG. 6.

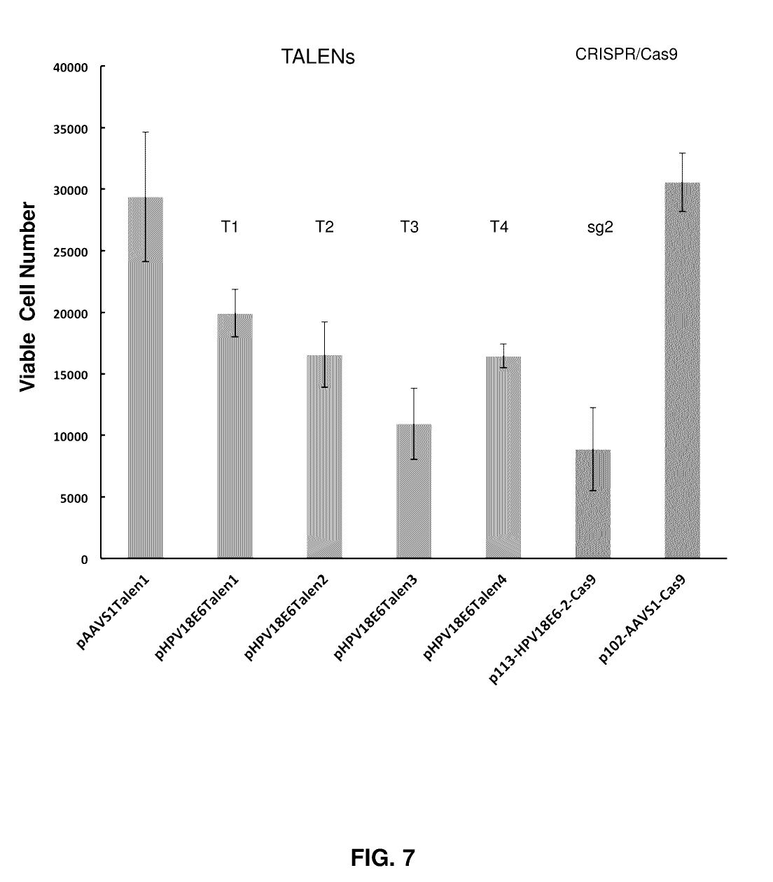

Viable cells were counted on day 5 for each of the transfected cell plates. Similar killing rates were observed with HPV 18 E6-specific TALEN (pHPV18E6Talen3) and CRISPR/Cas9 (p113-HPV18E6-2-Cas9). The viable cell counts for each of the TALENs and CRISPR/Cas9 complexes is shown in FIG. 7.

The AAVS1 site is present in the human genome and, as shown in FIG. 7, cleavage at AAVS1, unlike cleavage in the HPV 18 E6 region, does not kill cells as indicated by the increased cell counts on the plates containing cells transfected with pAAVS1Talen1 and p102-AAVS1-Cas9.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A composition comprising:
a targeting peptide that binds specifically to a specific viral nucleic acid, and
a cleaving peptide linked to the targeting peptide, wherein the cleaving peptide is the cleavage domain of a nuclease, and wherein the cleavage domain cleaves nucleic acid in a non-sequence specific manner.

2. The composition of claim 1, wherein the targeting peptide is a viral protein

3. The composition of claim 2, wherein the viral protein is selected from the group consisting of herpes simplex virus protein vmw65, EBNA-1, EBNA-2, EBNA-3, LMP-1, LMP-2 and EBER.

4. The composition of claim 1, wherein the nuclease comprises a Type IIS enzyme selected from the group consisting of Aar 1, BsrB I, SspD5 I, Ace III, BsrD I, Sth132 I, Aci I, BstF5 I, Sts I, AIo I, Btr I, TspDT I, Bae I, Bts I, TspGW I, Bbr7 I Cdi I Tth1 11II, Bbv I, CjeP I, UbaP I, Bbv II, Drd II, Bsa I, BbvC I, Eci I, BsmB I, Bed Eco31, Bce83 I, Eco57 I, BceAI, Eco57M I, Bcef I Esp3I, Beg I, Faul, BciVI, Fin I, BfiI, FokI, Bin I, GdiII, BmgI, GsuI, Bpul0I, HgaI, BsaXI, Hin4 II, BsbI, HphI, BscAI, Ksp632 I, BscGI, Mbo π, BseRI, MIyI, BseYI, MmeI, BsiI, MnII, BsmI, PfII, 108 I, BsmAI, PIeI, BsmFI, PpiI, Bsp24I, PsrI, BspGI, R1eAI, BspMI, Sap I, BspNC I, SfaNI, Bsr I, and Sim I.

5. The composition of claim 1, wherein the viral nucleic acid is viral DNA.

6. The composition of claim 5, wherein the viral DNA is DNA from a virus from the group consisting of herpes simplex virus (HSV)-1, HSV-2, varicella zoster virus (VZV), Epstein-Barr virus (EBV), cytomegalovirus (CMV), human herpesvirus (HHV)-6A and -6B, HHV-7, Kaposi's sarcoma-associated herpesvirus (KSHV).

7. The composition of claim 1, wherein the targeting peptide and the cleaving peptide are covalently linked.

8. The composition of claim 7, wherein the targeting peptide and the cleaving peptide are covalently linked by at least one peptide bond.

9. The composition of claim 1, wherein the cleaving peptide dimerizes with a second cleaving peptide.

10. The composition of claim 3, wherein the viral protein is EBNA1.

11. The composition of claim 4, wherein the nuclease comprises FokI.

12. The composition of claim 6, wherein the virus is the Epstein-Barr virus (EBV).

13. A composition comprising nucleic acid encoding:
a targeting peptide that binds specifically to a specific viral nucleic acid, and
a cleaving peptide linked to the targeting peptide, wherein the cleaving peptide is the cleavage domain of a nuclease, and wherein the cleavage domain cleaves nucleic acid in a non-sequence specific manner.

14. The composition of claim 13, wherein the nucleic acid is provided within a vector.

15. The composition of claim 14, wherein the vector is a plasmid.

16. The composition of claim 13, wherein the nucleic acid comprises mRNA.

17. The composition of claim 13, wherein the specific viral nucleic acid is viral DNA.

18. The composition of claim 17, wherein the viral DNA is DNA from a virus from the group consisting of herpes simplex virus (HSV)-1, HSV-2, varicella zoster virus (VZV), Epstein-Barr virus (EBV), cytomegalovirus (CMV), human herpesvirus (HHV)-6A and -6B, HHV-7, Kaposi's sarcoma-associated herpesvirus (KSHV).

19. The composition of claim 13, wherein the targeting peptide is a viral protein selected from the group consisting of herpes simplex virus protein vmw65, EBNA-1, EBNA-2, EBNA-3, LMP-1, LMP-2 and EBER.

20. The composition of claim 13, wherein the nuclease comprises a Type IIS enzyme selected from the group consisting of Aar 1, BsrB I, SspD5 I, Ace III, BsrD I, Sth132 I, Aci I, BstF5 I, Sts I, AIo I, Btr I, TspDT I, Bae I, Bts I, TspGW I, Bbr7 I Cdi I Tth1 11II, Bbv I, CjeP I, UbaP I, Bbv II, Drd II, Bsa I, BbvC I, Eci I, BsmB I, Bed Eco31, Bce83 I, Eco57 I, BceAI, Eco57M I, Bcef I Esp3I, Beg I, Faul, BciVI, Fin I, BfiI, FokI, Bin I, GdiII, BmgI, GsuI, Bpul0I, HgaI, BsaXI, Hin4 II, BsbI, HphI, BscAI, Ksp632 I, BscGI, Mbo π, BseRI, MIyI, BseYI, MmeI, BsiI, MnII, BsmI, PfII, 108 I, BsmAI, PIeI, BsmFI, PpiI, Bsp24I, PsrI, BspGI, R1eAI, BspMI, Sap I, BspNC I, SfaNI, Bsr I, and Sim I.


SEQUENCE LISTING (1 sequence)

SEQ ID NO: 1 - DNA, 240 bp, Human papillomavirus type 18
gaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgcc